(19) United States
(12) Patent Application Publication
(10) Pub. No.: US 2011/0174312 A1
(43) Pub. Date: Jul. 21, 2011

(54) Occlusive Plug

(76) Inventors: John Sanders Everingham, Sheffield (GB); Gilbert Marcus Filshie, Nottingham (GB); Bernard Sweeney, Thames Ditton (GB); Adam Laszlo Magos, Hertfordshire (GB)

(21) Appl. No.: 13/120,850

(22) PCT Filed: Oct. 15, 2009
(86) PCT No.: PCT/GB2009/002450

§ 371 Date: Mar. 24, 2011

(30) Foreign Application Priority Data
Oct. 15, 2008 (GB) 0818852.6

(51) Int. Cl. A61F 6/22

(52) U.S. Cl. 128/831

(57) ABSTRACT

A plug (1) for use in occluding a fallopian tube hysteroscopically comprising an elongate body (2) having an external thread (3) characterised in that the body and/or external thread has/have a roughened and/or pitted external surface (4), or in that the thread is fenestrated (5), or in that the leading end (6) of the elongate body (2) is provided with an extension nose (7) having a smooth external surface (8) and a rounded terminal end (9), or in that the leading end (6) of the elongate body (2) is provided with a conic helix (11) of the same hand as the external thread (3), generally forming a continuation of the external thread (3). The invention also includes a drive tool, for inserting a plug (1) incorporating an integral flexible hysteroscope, and comprising an outer sleeve housing a flexible drive rod adapted, at one end to engage, and apply torque to, the trailing end (12,13) of the elongate body (2) of the plug (1), with the other end of the drive rod being manually rotatable by the surgeon.

### FIELD OF THE INVENTION

This invention relates to an occlusive plug for use in occluding a fallopian tube hysteroscopically and hence effective as a means of sterilisation.

### BACKGROUND OF THE INVENTION

The principle of a threaded plug to be retained within the fallopian tube is known from WO 97/12569 which describes a plug in the form of an elongate body having an external thread, which plug, upon rotation, screws itself into the fallopian tube, the plug being self-retaining, and effective when in situ for purposes of sterilisation. Clearly the plug dimensions need to be selected according to the species to be sterilised, with the dual requirement of security against migration which might be caused by muscular tubal peristalsis, yet an ability to remove the plug at a later date should it be required to reverse the sterilisation procedure. In WO 2007/072004 is described an improved plug less prone to ejection and hence sterilisation failure, by providing a thread with a large root length and a large pitch. The proposal was to produce the plug in implantable grade polyethylene and various dimensions and ratios were advanced for thread forms to meet the practical requirements.

### OBJECT OF THE INVENTION

A basic object of the present invention is the provision of a further improved plug.

### Summary of a First Aspect of the Invention

According to a first aspect of the present invention there is provided a plug for use in occluding a fallopian tube hysteroscopically comprising an elongate body having an external thread, characterised in that the body and/or external thread has/have a roughened and/or pitted external surface.

### Advantages of a First Aspect of the Invention

After insertion of the plug, tissue within the lumen will bind onto the plug to provide enhanced plug location, and occlude the lumen, whereby the roughing and/or pitting reduces the possibility of post operative migration of the plug resulting from muscular tubal peristalsis.

### Summary of a Second Aspect of the Invention

According to a second aspect of the invention, of independent significance, there is provided a plug for use in occluding a fallopian tube hysteroscopically comprising an elongate body having an external thread, characterized in that the thread is fenestrated.

### Advantages of a Second Aspect of the Invention

The fenestration enables endosalpinx and muscular tissue growth through the holes in the thread to aid anchorage of the plug occluding the lumen. This would also reduce the possibility of post operative migration of the plug. Such tissue growth would of course make the plug more difficult to remove should reversal of sterilisation be required, at some future date, but it is believed the lumen would quickly regenerate.

### Summary of a Third Aspect of the Invention

According to a third aspect of the invention, of independent significance, there is provided a plug for use in occluding a fallopian tube hysteroscopically comprising an elongate body having an external thread, characterized in that the leading end of the elongate body is provided with an extension nose having a smooth external surface and a rounded terminal end.

### Advantages of a Third Aspect of the Invention

The extension nose constitutes a pilot nose providing a self centering capability as the Plug is inserted into the lumen. As the lumen is often tortuous this feature is most advantageous in reducing the risk of the plug creating a false passage, and hence being less effective in its role of sterilisation.

### Summary of a Fourth Aspect of the Invention

According to a fourth aspect of the invention, of independent significance, there is provided a plug for use in occluding a fallopian tube hysteroscopically comprising an elongate body having an external thread, characterized in that the leading end of the elongate body is provided with a conic helix of the same hand as the external thread, generally forming a continuation of the external thread.

### Advantages of a Fourth Aspect of the Invention

The pilot nose in the form of a conic helix constitutes a lumen finder assisting initial location in, and penetration of, the lumen.

### Summary of a Fifth Aspect of the Invention

According to a fifth aspect of the invention, of independent significance, there is provided with a rotary drive tool, for inserting a plug in accordance with any of the above four aspects hysteroscopically into a fallopian tube, the tool incorporating an integral flexible hysteroscope and comprising an outer sleeve housing a flexible drive rod adapted, at one end, to engage and apply torque to, the drive head of the plug, with the other end of the drive rod being manually rotatable by the surgeon.

### Preferred or Optional Features of the Various Aspects of the Invention

The plug is injection moulded in a rigid, inert, bio-compatible grade synthetic plastics.

The synthetic plastics is polyethylene.

The body and/or external thread is/are roughened and/or pitted during injection moulding.

The body and/or external thread is/are roughened and/or pitted by laser subsequently to moulding.

Roughening and/or pitting is up to 1 mm depth.

Fenestration is achieved by providing fine holes, approximately 1 mm diameter or less through the threads.

The extension nose is co-axial with the elongate body and is of circa 10 mm length.

The plug is of length 1.25 cm to 3.5 cm.

The plug is of diameter 2 mm to 5 mm.

The plug is formed at its trailing end as a drive head, which is adapted, in use, to be engaged by a portion of a drive tool.

The extension/pilot nose of the plug is engaged with the ostia of the fallopian tube/lumen to be occluded, by temporary attachment of the plug to one end of a drive tool, the latter incorporating an integral flexible hysteroscope and comprising an outer sleeve housing a flexible drive rod adapted, at one end to engage, and apply torque to, the trailing end of the elongate body of the plug, with the other end of the drive rod being manually rotatable by the surgeon.

### BRIEF DESCRIPTION OF THE DRAWINGS

The invention will now be described in greater detail, by way of examples with reference to the accompanying drawings, in which:

FIG. 1 is a perspective view to an enlarged scale, of a first embodiment of plug;

FIG. 2 is an enlargement of a portion of FIG. 1;

FIG. 3 shows the plug of FIGS. 1 and 2 after insertion;

FIG. 4 is a perspective view to an enlarged scale, of a second embodiment of plug;

FIG. 5 is an enlargement of a portion of FIG. 4;

FIG. 6 is a side elevation of the plug of FIGS. 4 and 5;

FIG. 7 is a perspective view to an enlarged scale, of a third embodiment of plug;

FIG. 8 shows the plug of FIG. 6, after insertion;

FIG. 9 is a perspective view to an enlarged scale, of a fourth embodiment of plug;

FIG. 10 shows a portion of FIG. 8; and

FIG. 11 shows a helix pilot portion.

In all embodiments, like parts are accorded like reference numerals.

A plug 1 for use in occluding a fallopian tube hysteroscopically comprises an elongate body 2 having an external thread 3, preferably being 1.25 cm to 3.5 cm in length, and 2 mm to 5 mm in diameter.

The plug 1 is injection moulded in a rigid, inert, bio-compatible synthetic plastics grade polyethylene.

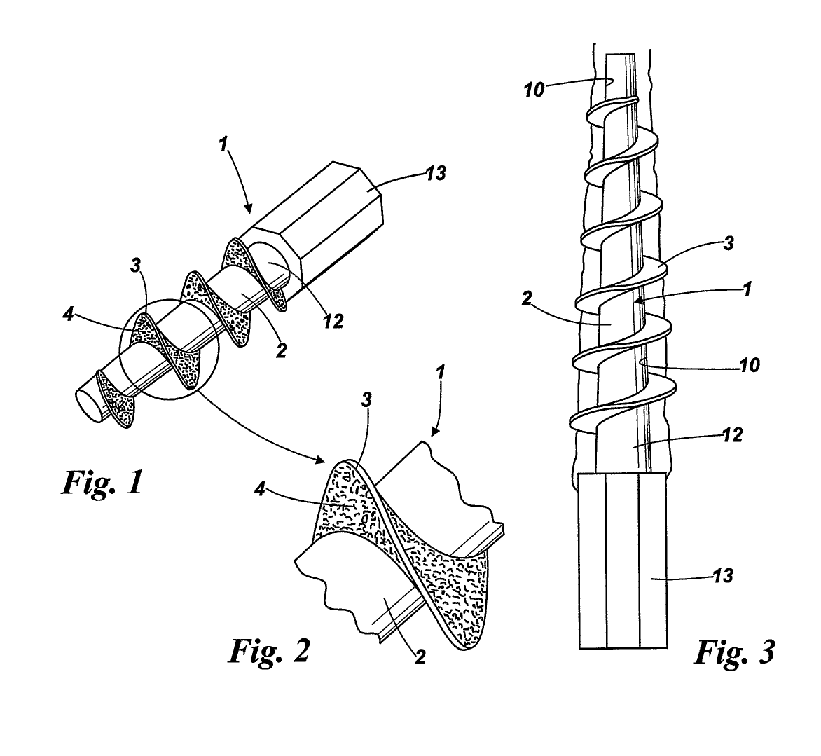

In accordance with the first embodiment illustrated in FIGS. 1 to 3, the body 2 and/or external thread 3 is roughened or pitted at 4.

The roughing or pitting is effected by a laser after injection moulding to a depth <1 mm.

Of course, instead of pitting an injection moulded plug e.g. by laser, the roughening of an otherwise smooth surface could be achieved by pitting the surfaces of the mould tools e.g. by spark erosion, so that projections of polyethylene extend from the body 2 and/or thread 3 by a height of <1 mm.

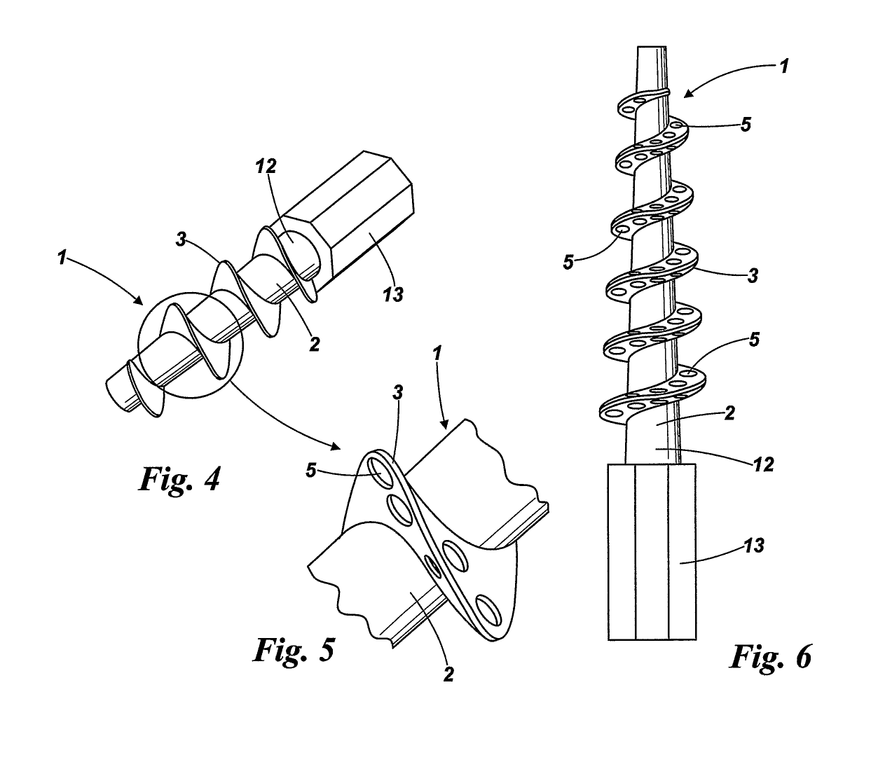

In the second embodiment, illustrated in FIGS. 4 to 6, pitting of the thread 3 is effective to such an extent that the thread 3 is pierced or perforated by the formation of micropores 5 of e.g. 0.01 mm to 0.1 mm diameter.

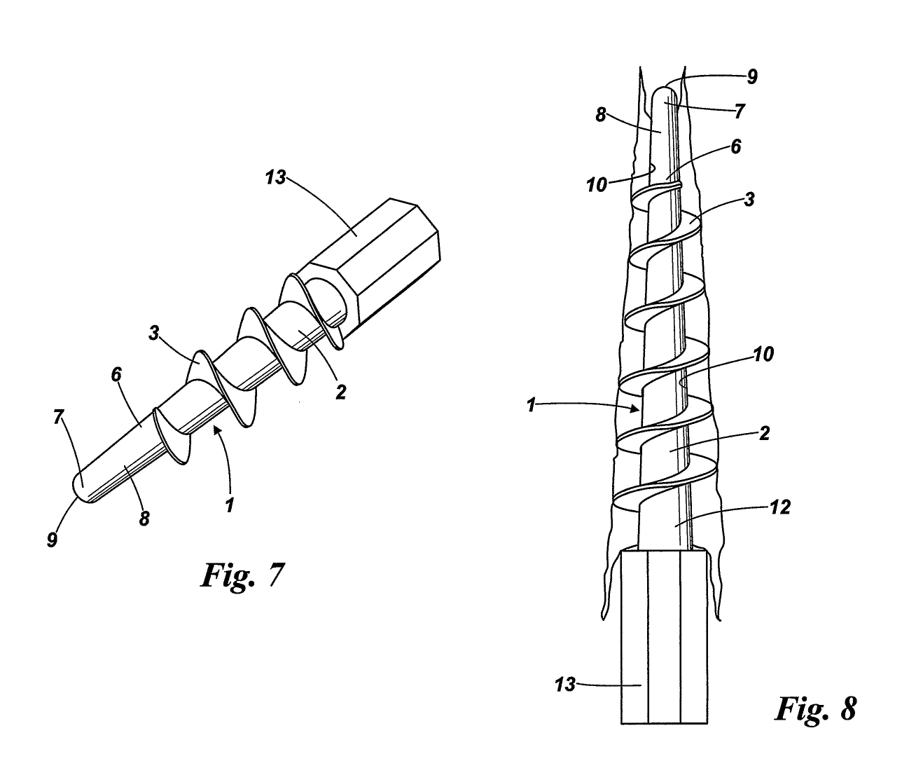

In the third embodiment, illustrated in FIGS. 7 and 8, leading end 6 of the elongate body 2 is provided with an extension nose 7 having a smooth external surface 8 and a rounded terminal end 9 to assist penetration of the lumen 10.

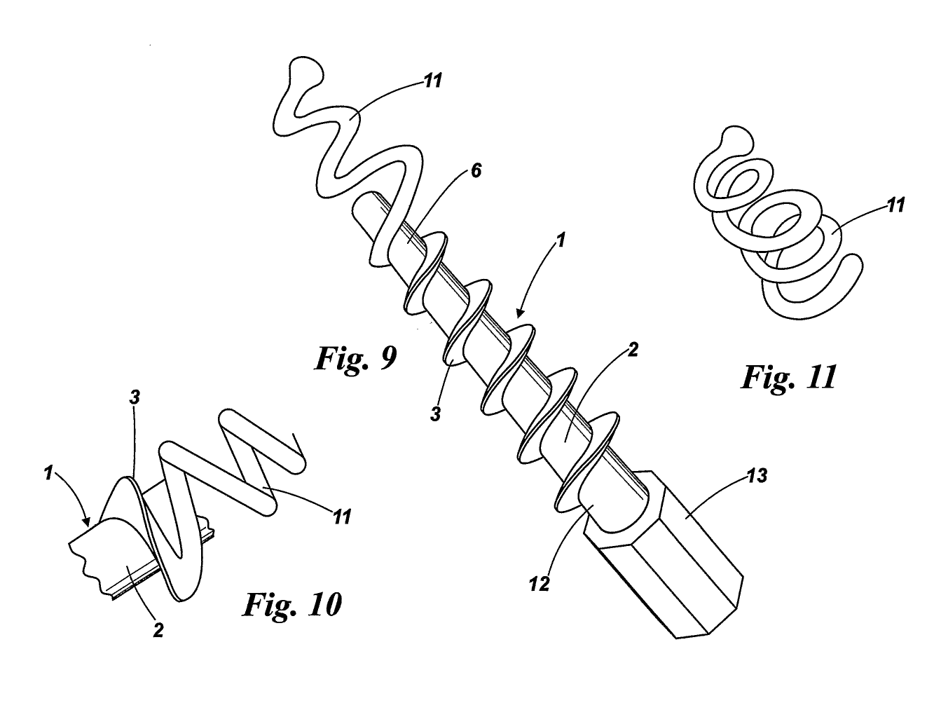

In the fourth embodiment, illustrated in FIGS. 9 to 11, the leading end 6 is provided with a conic helix 11 of the same hand as the thread 3 and generally forms a continuation of the thread 3.

At its trailing end 12, the plug 1 is provided a hexagonal drive head 13 for temporary attachment to a suitable connector of rotary drive tool (not shown) incorporating an integral flexible hysteroscope, the leading end 6 of the plug 1 being engaged with the ostia of a fallopian tube to be occluded and the plug 1 then slowly rotated by the surgeon, to screw the plug 1 into and along the lumen 10, the drive tool basically comprising an outer sleeve housing a flexible drive rod adapted to be drivably engageable with, and readily releasably from, the drive head 13 of a plug 1, after completion of insertion.

1. A plug for use in reversibly occluding a fallopian tube hysteroscopically comprising an elongate body having an external thread characterised in that the body and/or external thread has/have a roughened and/or pitted external surface.

2. A plug for use in reversibly occluding a fallopian tube hysteroscopically comprising an elongate body having an external thread, characterised in that the thread is fenestrated.

3. A plug for use in reversibly occluding a fallopian tube hysteroscopically comprising an elongate body having an external thread, characterised in that the leading end of the elongate body is provided with an extension nose having a smooth external surface and a rounded terminal end.

4. (canceled)

5. A plug, as claimed in claim 1, which is injection moulded in a rigid, inert, bio-compatible grade synthetic plastics.

6. A plug as claimed in claim 5, wherein the plastic is polyethylene.

7. A plug as claimed in claim 5, wherein the body and/or external thread is/are roughened and/or pitted during injection moulding.

8. A plug as claimed in claim 5, wherein the body and/or external thread is/are roughened and/or pitted by laser subsequently to injection moulding.

9. A plug as claimed in claim 1, wherein roughening and/or pitting is up to 1 mm depth.

10. A plug as claimed in claim 2, wherein fenestration is achieved by providing fine holes, approximately 1 mm diameter or less through the threads.

11. A plug as claimed in claim 3, wherein the extension nose is co-axial with the elongate body and is of circa 10 mm length.

12. A plug as claimed in claim 1, of length 1.25 cm to 3.5 cm.

13. A plug as claimed in claim 1, of diameter 2 mm to 5 mm.

14. A plug as claimed in claim 1, formed at its trailing end as a drive head, which is adapted, in use, to be engaged by a portion of a drive tool.

15. A rotary drive tool, for inserting a plug as defined in claim 1 hysteroscopically into a fallopian tube, the tool incorporating an integral flexible hysteroscope and comprising an outer sleeve housing a flexible drive rod adapted, at one end, to engage and apply torque to, the drive head of the plug, with the other end of the drive rod being manually rotatable by the surgeon.

16. A plug, as claimed in claim 2, which is injection moulded in a rigid, inert, bio-compatible grade synthetic plastics.

17. A plug as claimed in claim 16, wherein the plastic is polyethylene.

18. A plug, as claimed in claim 3, which is injection moulded in a rigid, inert, bio-compatible grade synthetic plastics.

19. A plug as claimed in claim 18, wherein the plastic is polyethylene.

20. A plug as claimed in claim 2, of length 1.25 cm to 3.5 cm.

21. A plug as claimed in claim 3, of length 1.25 cm to 3.5 cm.

22. A plug as claimed in claim 2, of diameter 2 mm to 5 mm.

23. A plug as claimed in claim 3, of diameter 2 mm to 5 mm.

24. A plug as claimed in claim 2, formed at its trailing end as a drive head, which is adapted, in use, to be engaged by a portion of a drive tool.

25. A plug as claimed in claim 3, formed at its trailing end as a drive head, which is adapted, in use, to be engaged by a portion of a drive tool.

26. A rotary drive tool, for inserting a plug as defined in claim 2 hysteroscopically into a fallopian tube, the tool incorporating an integral flexible hysteroscope and comprising an outer sleeve housing a flexible drive rod adapted, at one end, to engage and apply torque to, the drive head of the plug, with the other end of the drive rod being manually rotatable by the surgeon.

27. A rotary drive tool, for inserting a plug as defined in claim 3 hysteroscopically into a fallopian tube, the tool incorporating an integral flexible hysteroscope and comprising an outer sleeve housing a flexible drive rod adapted, at one end, to engage and apply torque to, the drive head of the plug, with the other end of the drive rod being manually rotatable by the surgeon.
